(19) United States
(12) Patent Application Publication
(10) Pub. No.: US 2025/0281353 A1
(43) Pub. Date: Sep. 11, 2025

(54) FOOT MASSAGER

(71) Applicant: FUJIAN ROVOS FITNESS CO., LTD., NINGDE (CN)

(72) Inventors: JINGHUA WU, NINGDE (CN); RONGMIN CHEN, NINGDE (CN)

(21) Appl. No.: 19/218,655

(22) Filed: May 27, 2025

Related U.S. Application Data
(63) Continuation of application No. PCT/CN2023/098451, filed on Jun. 6, 2023.

(30) Foreign Application Priority Data
Nov. 30, 2022 (CN) 202211530463.X

(51) Int. Cl. A61H 23/02

(52) U.S. Cl. CPC A61H 23/0263; A61H 2023/0281; A61H 2201/0169; A61H 2201/0207; A61H 2201/1454; A61H 2201/164; A61H 2201/5048; A61H 2201/5097; A61H 2205/12

(57) ABSTRACT

A foot massager includes an upper cover assembly, a lower cover assembly, and a foot massage assembly. The foot massage assembly is suspended in the lower cover assembly by fasteners. The foot massage assembly realizes a vibration function and a heating function. In realizing the vibration function, connecting rods thereof are symmetrically disposed, and counterweight blocks are introduced to realize weight balance, thereby realizing a stable mechanism movement of the foot massage assembly. In each of the heating assemblies, a heat-conducting copper sheet thereof conducts heat of a heating sheet thereof to a corresponding one of massage heads. The foot massage assembly is connected to the upper cover assembly through the massage heads and upper connecting rods. The massage heads are assembled last. Foot pads disposed on the upper cover assembly are respectively pressed against the massage heads through the upper connecting rods.

### TECHNICAL FIELD

The present disclosure relates to a technical field of pedicure machines, and in particular to a foot massager.

### BACKGROUND

Most foot massagers on the market have a problem of excessive noise, especially when using a strong gear to massage feet of a user, which greatly affects the user experience.

In addition, most of eccentric wheels of the foot massagers on the market have only one eccentric direction when massaging the feet. When a motor of the foot massagers works, the eccentric wheels rotate, and a gravity center of each of the eccentric wheels changes around a motor shaft in the eccentric direction. The gravity center of each of the eccentric wheels is not on the motor shaft, and the farther the gravity center of each of the eccentric wheels is from the motor shaft, the greater the amplitude of each of the eccentric wheels vibrates, and the greater the vibration generated. Therefore, it is necessary to provide a foot massager with no vibration or slight vibration. Moreover, it is necessary to provide a foot massager where massage heads thereof have a heating function.

### SUMMARY

The present disclosure provides a foot massager to solve problems in the prior art.

The foot massager comprises an upper cover assembly, a lower cover assembly, and a foot massage assembly. The upper cover assembly is fixed to the lower cover assembly through screws. The foot massage assembly is disposed between the upper cover assembly and the lower cover assembly. The foot massage assembly comprises a brushless motor, a motor shaft, eccentric wheels, upper connecting rods, lower connecting rods, counterweight blocks and massage heads. The motor shaft is rotatably connected to the brushless motor. The eccentric wheels are sleeved on the motor shaft. Each of the eccentric wheels comprises a first eccentric section and a second eccentric section. An eccentric direction of each first eccentric section is opposite to an eccentric direction of each second eccentric section. Each of the upper connecting rods is installed on a corresponding first eccentric section. Each of the lower connecting rods is installed on a corresponding second eccentric section. Each of the massage heads is installed on an upper end of a corresponding one of the upper connecting rods. Each of the counterweight blocks is installed on a lower end of a corresponding one of the lower connecting rods.

Optionally, the upper cover assembly comprises an upper cover, a top cover, a left foot pad, a right foot pad, a control panel, a display patch, and a light guiding piece. The foot massager further comprises an ornament and a stainless steel trim.

The upper cover is installed on an upper end of the lower cover assembly. The top cover is buckled with the upper cover. The left foot pad and the right foot pad are installed in the top cover. The control panel and the display patch are sequentially disposed on a top surface of the top cover from bottom to top. The light guiding piece is disposed around the display patch. The ornament and the stainless steel trim are installed on one side of the upper cover assembly. The stainless steel trim defines round holes configured to transmit sound.

Optionally, the lower cover assembly comprises a lower cover, a shockproof base, anti-friction pads, a main board, a BLUETOOTH board, a speaker, and a direct current (DC) power socket. The lower cover is buckled with the upper cover. The shockproof base is installed on a lower surface of the lower cover. The shockproof base is of an integrally formed structure and is made from rubber. The anti-friction pads are installed on a bottom portion of the shockproof base. The main board, the BLUETOOTH board, the speaker, and the DC power socket are installed on the lower cover.

Optionally, the foot massage assembly further comprises a motor seat, a motor fixing piece, vibration mechanisms, bearing seats, connecting rod rubber pieces, and heating assemblies. The brushless motor is installed on the motor seat through the motor fixing piece. The vibration mechanisms are installed on the motor seat. Two ends of the motor shaft of the brushless motor are respectively rotatably connected to the bearing seats. The bearing seats are respectively installed in the vibration mechanisms. Upper ends of the upper connecting rods and lower ends of the lower connecting rods are respectively installed in the vibration mechanisms through the connecting rod rubber pieces. The heating assemblies are respectively installed on the upper ends of the upper connecting rods. The heating assemblies are respectively located at lower portions of the massage heads.

Optionally, each of the heating assemblies comprises an upper heating sheet fixing piece, a heat-conducting copper sheet, a middle heating sheet fixing piece, a heating sheet, and a lower heating sheet fixing piece.

In each of the heating assemblies, the middle heating sheet fixing piece thereof is fixed on a corresponding one of the upper connecting rods, the heating sheet thereof is fixed to the middle heating sheet fixing piece thereof, the heat-conducting copper sheet thereof is disposed on the heating sheet thereof, the upper heating sheet fixing piece thereof is disposed between the heating sheet thereof and a corresponding one of the massage heads, and the lower heating sheet fixing piece thereof is disposed between the heating sheet thereof and a corresponding one of the connecting rod rubber pieces.

Optionally, the foot massage assembly further comprises rubber fixing pieces. The connecting rod rubber pieces respectively disposed on the upper connecting rods are respectively installed on upper portions of the vibration mechanisms through first rubber fixing pieces. The connecting rod rubber pieces respectively disposed on the lower connecting rods are respectively installed on lower portions of the vibration mechanisms through second rubber fixing pieces.

Optionally, the foot massage assembly further comprises bearing rubber sleeves and motor bearings. The two ends of the motor shaft are respectively installed in the bearing seats through the motor bearings. Each of the bearing rubber sleeves is sleeved on an outer ring of a corresponding one of the motor bearings and are sleeved in an inner hole of a corresponding one of the bearing seats.

Optionally, the foot massage assembly further comprises sealed bearings, first bearing fixing pieces, second bearing fixing pieces, and pressing plates. The sealed bearings comprise first sealed bearings and second sealed bearings.

Each of the upper connecting rods is installed on the corresponding first eccentric section through corresponding first sealed bearings, and a first pressing plate of the pressing plates presses the corresponding first sealed bearings disposed in each of the upper connecting rods on an inner side surface of each of the upper connecting rods.

Each of the lower connecting rods is installed on the corresponding second eccentric section through corresponding second sealed bearings. The corresponding second sealed bearings are disposed in each of the lower connecting rods, and a second pressing plate of the pressing plates presses the corresponding second sealed bearings on an inner side surface of each of the lower connecting rods.

Each of the first bearing fixing pieces abuts against inner side surfaces of the corresponding first sealed bearings disposed in each of the upper connecting rods. Each of the second bearing fixing pieces abuts against inner side surfaces of corresponding second sealed bearings disposed in each of the lower connecting rods.

Optionally, the foot massage assembly further comprises elastic pins. The eccentric wheels are limited on the motor shaft through the elastic pins respectively.

Optionally, the foot massage assembly further comprises fasteners, the fasteners being integrally formed rubber structures. The fasteners are respectively installed on end portions of the vibration mechanisms. The fasteners are hung in the lower cover.

In the foot massager, the upper connecting rods and the lower connecting rods are symmetrically disposed, and the counterweight blocks are introduced to realize weight balance, so as to realize a stable massage operation and effectively reduce the vibration. Therefore, the foot massager only produces a slight vibration or almost no vibration is produced. In addition, a noise generated by the massage operation of the foot massager is very small. Specifically, the foot massage assembly is suspended on the lower cover assembly by the fasteners that are flexible, and the shockproof base of the lower cover assembly is a flexible integrated structure, which is able to absorb vibration to the maximum extent and reduces the noise. A heating function of the foot massage assembly is realized by each heat-conducting copper sheet directing the heat of each heating sheet to each of the massage heads, and the upper heating sheet fixing piece and the lower heating sheet fixing piece of each of the heating assemblies isolate each heating sheet from contact with other objects. The foot massager has a simple structure, simple assembly, and low cost. Specifically, the massage heads of the foot massage assembly are respectively connected to screw threads of the upper connecting rods, and the massage heads respectively press the foot pads disposed on the upper cover assembly to realize a connection between the foot massage assembly and the upper cover assembly. The foot pads are flexible, have a dust-proof effect, and prevent the massage heads from loosening.

### BRIEF DESCRIPTION OF DRAWINGS

In order to clearly describe technical solutions in the embodiments of the present disclosure, the following will briefly introduce the drawings that need to be used in the description of the embodiments or the prior art. Apparently, the drawings in the following description are merely some of the embodiments of the present disclosure, and those skilled in the art are able to obtain other drawings according to the drawings without contributing any inventive labor.

It is noted that structures, proportions, sizes, and the like shown in the drawings of the present specification are only used to cooperate with the contents disclosed in the description, so as to be understood and read by those skilled in the art, and are not intended to limit the limiting conditions that can be implemented by the present disclosure, so that the technical content disclosed in the present disclosure should still fall within the scope covered by the technical content disclosed in the present disclosure without affecting the effect and the purpose that can be realized in the present disclosure.

FIG. 1 is an exploded schematic diagram of a foot massager according to one embodiment of the present disclosure.

FIG. 2 is a perspective schematic diagram of the foot massager according to one embodiment of the present disclosure.

FIG. 3 is an exploded schematic diagram of an upper cover assembly according to one embodiment of the present disclosure.

FIG. 4 is an exploded schematic diagram of a lower cover assembly according to one embodiment of the present disclosure.

FIG. 5 is a cross-sectional schematic diagram of foot massage assembly according to one embodiment of the present disclosure.

FIG. 6 is another cross-sectional schematic diagram of foot massage assembly according to one embodiment of the present disclosure.

FIG. 7 is a perspective schematic diagram of foot massage assembly according to one embodiment of the present disclosure.

FIG. 8 is an exploded schematic diagram of foot massage assembly according to one embodiment of the present disclosure.

In the figure: 100-upper cover assembly, 200-lower cover assembly, 300-foot massage assembly; 101-upper cover, 102-top cover, 103-left foot pad, 104-right foot pad, 105-control panel, 106-display patch, 107-light guiding piece, 108-ornament, 109-stainless steel trim; 201-lower cover, 202-shockproof base, 203-anti-friction pad, 204-main board, 205-BLUETOOTH board, 206-speaker, 207-DC power socket; 1-motor base, 2-brushless motor, 3-motor fixing piece, 4-motor shaft, 5-vibration mechanism, 6-eccentric wheel, 7-upper connecting rod, 8-lower connecting rod, 9-counterweight block, 10-massage head, 11-connecting rod rubber piece, 12-rubber fixing piece, 13-upper heating sheet fixing piece, 18-bearing seat, 19-motor bearing, 20-bearing rubber sleeve, 21-sealed bearing, 22-pressing plate, 23-first bearing fixing piece, 24-second bearing fixing piece, 25-elastic pin, 26-fastener.

### DETAILED DESCRIPTION

The following is a description of the implementation methods of the present disclosure by specific embodiments. Those skilled in the art can easily understand other characteristics and effects of the present disclosure from the contents disclosed in the specification. Obviously, the described embodiments are part of the embodiments of the present disclosure, and are not all of the embodiments. Based on the embodiments of the present disclosure, all other embodiments obtained by those skilled in the art without creative work are within the protection scope of the present disclosure.

### Embodiment 1

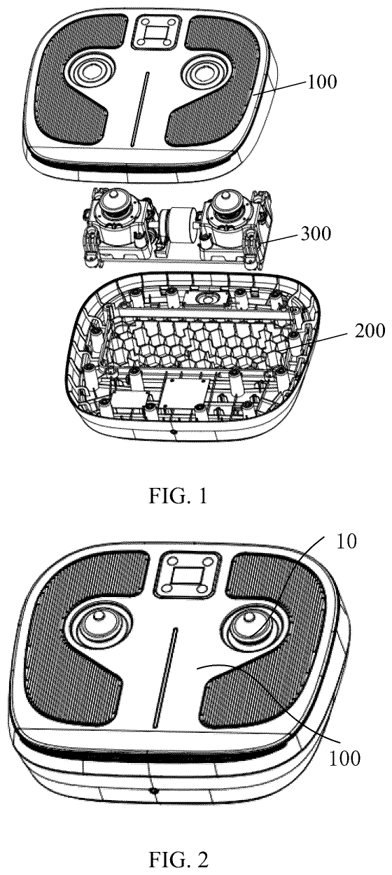

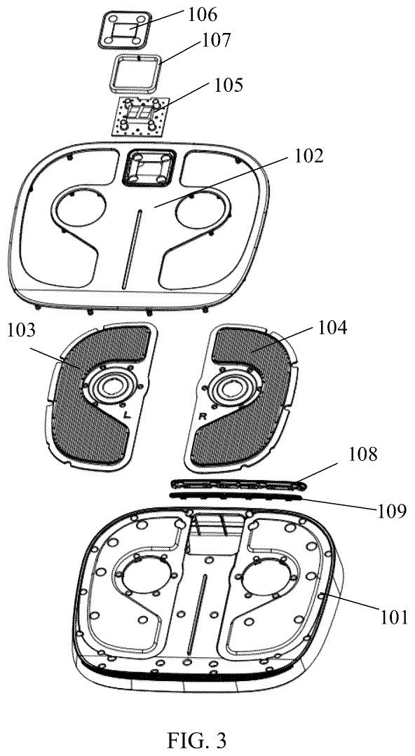

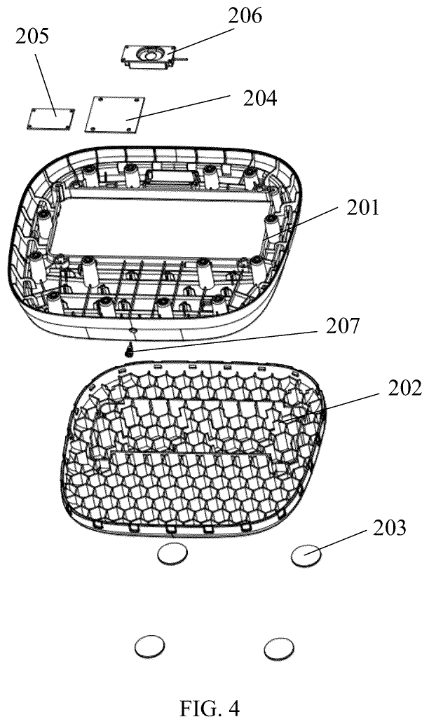

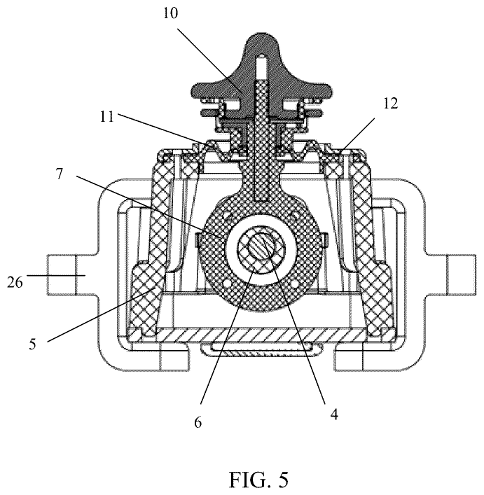

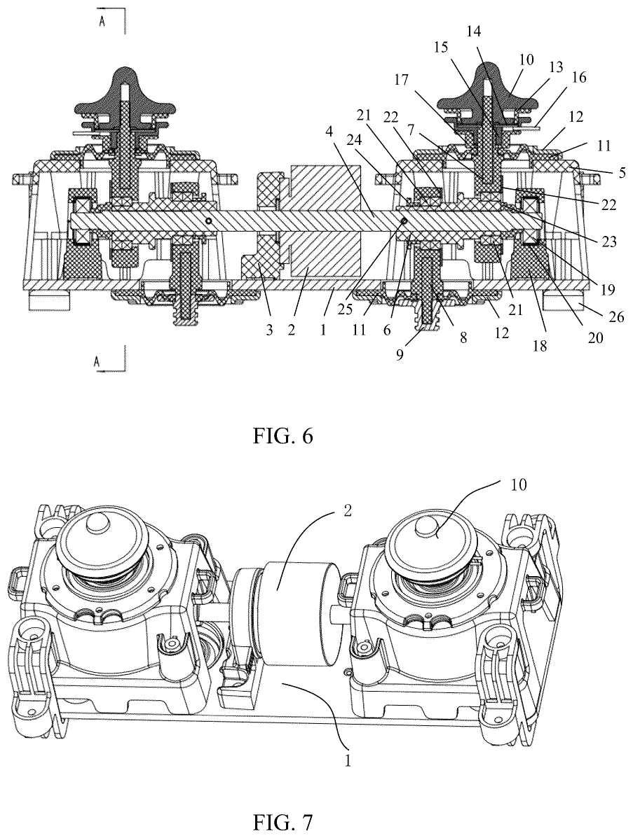

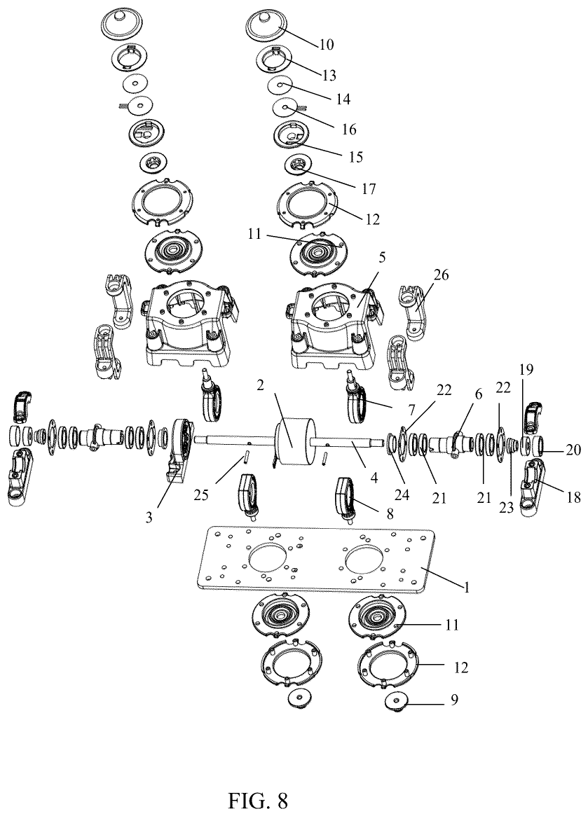

As shown in FIGS. 1-8, in a first aspect, the present disclosure provides a foot massager. The foot massager comprises an upper cover assembly 100, a lower cover assembly 200, and a foot massage assembly 300. The upper cover assembly 100 is fixed to the lower cover assembly 200 through screws. The foot massage assembly 300 is disposed between the upper cover assembly 100 and the lower cover assembly 200. The foot massage assembly 300 comprises a brushless motor 2, a motor shaft 4, eccentric wheels 6, upper connecting rods 7, lower connecting rods 8, counterweight blocks 9 and massage heads 10. The motor shaft 4 is rotatably connected to the brushless motor 2. The eccentric wheels 6 are sleeved on the motor shaft 4. Each of the eccentric wheels 6 comprises a first eccentric section and a second eccentric section. An eccentric direction of each first eccentric section is opposite to each eccentric direction of the second eccentric section. Each of the upper connecting rods 7 is installed on a corresponding first eccentric section. Each of the lower connecting rods 8 is installed on a corresponding second eccentric section. Each of the massage heads 10 is installed on an upper end of a corresponding one of the upper connecting rods 7. Each of the counterweight blocks 9 is installed on a lower end of a corresponding one of the lower connecting rods 8.

It should be noted that the foot massage assembly 300 is connected to the lower cover assembly 200 by structures made from a flexible material. The foot massage assembly 300 is connected to the upper cover assembly 100 by pressing the massage heads 10 and the upper connecting rods 7 of the foot massage assembly 300 against the foot pads disposed on two sides of the upper cover assembly 100.

In the foot massager, the upper connecting rods 7 and the lower connecting rods 8 are symmetrically disposed, and the counterweight blocks 9 are introduced to realize weight balance, so as to realize a stable massage operation and effectively reduce the vibration. Therefore, the foot massager only produces a slight vibration or almost no vibration is produced, which reduces a noise impact generated by the movement of the foot massage assembly is reduced. Furthermore, a gravity center deviation of the foot massager is balanced by the counterweight blocks 9 when the foot massager massages feet, so that a gravity center of each of the eccentric wheels 6 is close to a center thereof when the eccentric wheels 6 rotate.

### Embodiment 2

As shown in FIGS. 1-8, in a second aspect, the present disclosure provides a foot massager, and the foot massager includes all of structures of the foot massager of the embodiment 1. Furthermore, the upper cover assembly 100 comprises an upper cover 101, a top cover 102, a left foot pad 103, a right foot pad 104, a control panel 105, a display patch 106, and a light guiding piece 107. The foot massager further comprises an ornament 108 and a stainless steel trim 109. The upper cover 101 is installed on an upper end of the lower cover assembly 200. The top cover 102 is buckled with the upper cover 101. The left foot pad 103 and the right foot pad 104 are installed in the top cover 102. The control panel 105 and the display patch are sequentially disposed on a top surface of the top cover 102 from bottom to top. The light guiding piece 107 is disposed around the display patch 106. The ornament 108 and the stainless steel trim 109 are installed on one side of the upper cover assembly 100. The stainless steel trim 109 defines round holes configured to transmit sound.

Optionally, the lower cover assembly 200 comprises a lower cover 201, a shockproof base 202, anti-friction pads 203, a main board 204, a BLUETOOTH board 205, a speaker 206, and a direct current (DC) power socket 207. The lower cover 201 is buckled with the upper cover 101. The shockproof base 202 is installed on a lower surface of the lower cover 201. The shockproof base 202 is of an integrally formed structure and is made from rubber. The anti-friction pads 203 are installed on a bottom portion of the shockproof base 202. The main board 204, the BLUETOOTH board 205, the speaker 206, and the DC power socket are installed on the lower cover 201.

In the foot massager of the embodiment, the top cover 102, the left foot pad 103, the right foot pad 104, and the upper cover 101 of the upper cover assembly 100 constitute main visible parts of the upper cover assembly 100. The left foot pad 103 and the right foot pad 104 made from silicone provide the user with a comfortable use experience. The control panel 105, the display patch 106, and the light guiding piece 107 together constitute a human-computer interaction control panel. The light guiding piece 107 makes the control panel beautiful. The ornament 108 and the stainless steel trim 109 disposed on one side of the upper cover assembly 100 play a decorative role. Moreover, the stainless steel trim 109 with the round holes further plays a role of a sound outlet, thereby reducing the noise impact generated by the movement of the foot massage assembly 300.

Main visible parts of the lower cover assembly 200 are the lower cover 201 and the shockproof base 202. The shockproof base 202 is made from the silicone, which avoids hard contact between the foot massager and the ground, absorbs most of the vibrations generated by the movement of the foot massage assembly 300, and reduces the generation of noise. The main board 204, the BLUETOOTH board 205, the speaker 206, and the DC power socket 207 are fixed on the lower cover 201, so that the foot massager has a music playback function. Four anti-friction pads 203 are disposed on the bottom portion of the shockproof base 202. The shockproof base 202 in the lower cover assembly 200 is made from silicone, which further absorbs vibrations and reduces the noise generated by the contact between the foot massager and the ground. In this way, after multiple-level vibration absorption, the noise is greatly reduced. The massage heads 10 of the foot massage assembly are respectively connected to screw threads of the upper connecting rods 7, and the massage heads 10 respectively press against the foot pads disposed on the upper cover assembly 100 to realize a connection between the foot massage assembly and the upper cover assembly 100. The foot pads are flexible, have a dust-proof effect, and prevent the massage heads 10 from loosening.

### Embodiment 3

As shown in FIGS. 1-8, in a third aspect, the present disclosure provides a foot massager, and the foot massager includes all of structures of the foot massager of the embodiment 2. Furthermore, the foot massage assembly 300 further comprises a motor seat 1, a motor fixing piece 3, vibration mechanisms 5, bearing seats 18, connecting rod rubber pieces 11, and heating assemblies. The brushless motor 2 is installed on the motor seat 1 through the motor fixing piece 3. The vibration mechanisms 5 are installed on the motor seat 1. Two ends of the motor shaft 4 of the brushless motor 2 are respectively rotatably connected to the bearing seats 18. The bearing seats 18 are respectively installed in the vibration mechanisms 5. Upper ends of the upper connecting rods 7 and lower ends of the lower connecting rods 8 are respectively installed in the vibration mechanisms 5 through the connecting rod rubber pieces 11. The heating assemblies are respectively installed on the upper ends of the upper connecting rods 7. The heating assemblies are respectively located at lower portions of the massage heads 10.

### Embodiment 4

As shown in FIGS. 1-8, in a fourth aspect, the present disclosure provides a foot massager, and the foot massager includes all of structures of the foot massager of the embodiment 3. Furthermore, each of the heating assemblies comprises an upper heating sheet fixing piece 13, a heat-conducting copper sheet 14, a middle heating sheet fixing piece 15, a heating sheet 16, and a lower heating sheet fixing piece 17.

In each of the heating assemblies, the middle heating sheet fixing piece 15 thereof is fixed on a corresponding one of the upper connecting rods 7, the heating sheet 16 thereof is fixed to the middle heating sheet fixing piece 15 thereof, the heat-conducting copper sheet 14 thereof is disposed on the heating sheet 16 thereof, the upper heating sheet fixing piece 13 thereof is disposed between the heating sheet 16 thereof and a corresponding one of the massage heads 10, and the lower heating sheet fixing piece 17 thereof is disposed between the heating sheet 16 thereof and a corresponding one of the connecting rod rubber pieces 11.

In each of the heating assemblies of the foot massager of the embodiment, the heat-conducting copper sheet 14 thereof conducts heat generated by the heating sheet 16 thereof to the corresponding one of massage heads to heat the feet of the user. In each of the heating assemblies, the upper heating sheet fixing piece 13 thereof isolates the heating sheet 16 thereof from a corresponding foot pad, so that the heating sheet 16 thereof does not directly contact the corresponding foot pad. In each of the heating assemblies, the lower heating sheet fixing piece 17 thereof isolates the heating sheet 16 thereof to prevent the heating sheet thereof from directly contacting the corresponding one of the connecting rod rubber pieces 11.

### Embodiment 5

As shown in FIGS. 1-8, in a fifth aspect, the present disclosure provides a foot massager, and the foot massager includes all of structures of the foot massager of the embodiment 4. Furthermore, the foot massage assembly 300 further comprises rubber fixing pieces 12. The connecting rod rubber pieces 11 respectively disposed on the upper connecting rods 7 are respectively installed on upper portions of the vibration mechanisms 5 through first rubber fixing pieces 12. The connecting rod rubber pieces 11 respectively disposed on the lower connecting rods 8 are respectively installed on lower portions of the vibration mechanisms 5 through second rubber fixing pieces 12.

### Embodiment 6

As shown in FIGS. 1-8, in a sixth aspect, the present disclosure provides a foot massager, and the foot massager includes all of structures of the foot massager of the embodiment 5. Furthermore, the foot massage assembly 300 further comprises bearing rubber sleeves 20 and motor bearings 19. The two ends of the motor shaft 4 are respectively installed in the bearing seats 18 through the motor bearings 19. The motor bearings 19 are 608ZZ bearings. Each of the bearing rubber sleeves 20 is sleeved on an outer ring of a corresponding one of the motor bearings 19 and are sleeved in an inner hole of a corresponding one of the bearing seats 18.

The motor bearings 19 respectively disposed on the two ends of the motor shaft of the brushless motor 2 are respectively sleeved in the bearing rubber sleeves 20. Since the motor shaft of the brushless motor 2 is too long, and there are some precision errors in processing or assembly technology, the brushless motor 2 and the two ends of the motor shaft may not be completely coaxial. If the two ends of the motor shaft are rigidly fixed, when the brushless motor 2 works, the two ends of the motor shaft 4 may generate opposite forces, which increase the noise generated by the movement of the brushless motor 2. The bearing rubber sleeves 20 that are flexible absorb the noise. The vibration generated when the foot massager massages the feet is acted on the motor shaft of the brushless motor 2 through the upper connecting rods 7. The flexible bearing rubber sleeves 20 disposed on the two ends of the motor shaft 4 further absorb vibration to the maximum extent and reduce the noise.

### Embodiment 7

As shown in FIGS. 1-8, in a seventh aspect, the present disclosure provides a foot massager, and the foot massager includes all of structures of the foot massager of the embodiment 6. Furthermore, the foot massage assembly 300 further comprises sealed bearings 21 and pressing plates 22. The sealing bearings 21 are 6803ZZ bearings. The sealed bearings 21 comprise first sealed bearings 21 and second sealed bearings 21. Each of the upper connecting rods 7 is installed on the corresponding first eccentric section through corresponding first sealed bearings 21, and a first pressing plate of the pressing plates 22 presses the corresponding first sealed bearings 21 disposed in each of the upper connecting rods 7 on an inner side surface of each of the upper connecting rods 7. Each of the lower connecting rods 8 is installed on the corresponding second eccentric section through corresponding second sealed bearings 21, the corresponding second sealed bearings 21 are disposed in each of the lower connecting rods 8, and a second pressing plate of the pressing plates 22 presses the corresponding second sealed bearings 21 on an inner side surface of each of the lower connecting rods 8.

Optionally, the foot massage assembly 300 further comprises first bearing fixing pieces 23, second bearing fixing pieces 24, and pressing plates 22. Each of the first bearing fixing pieces 23 abuts against inner side surfaces of the corresponding first sealed bearings 21 disposed in each of the upper connecting rods 7. The first bearing fixing pieces 23 facilitate the installation and disassembly of the corresponding first sealed bearings 21.

Optionally, the foot massage assembly 300 further comprises second bearing fixing pieces 24. Each of the second bearing fixing pieces abuts against inner side surfaces of corresponding second sealed bearings 21 disposed in each of the lower connecting rods 8. The second bearing fixing pieces 24 facilitate the installation and disassembly of the corresponding second sealed bearings 21.

Optionally, the foot massage assembly 300 further comprises elastic pins 25. The eccentric wheels 6 are limited on the motor shaft 4 through the elastic pins 25 respectively. The elastic pins 25 play a good position limiting function.

Optionally, the foot massage assembly 300 further comprises fasteners 26, the fasteners 26 are integrally formed rubber structures. The fasteners 26 are respectively installed on end portions of the vibration mechanisms 5. The fasteners 26 are hung in the lower cover 201. The foot massage assembly and the lower cover assembly are fixed through four fasteners 26 which are made from flexible rubber material. In this way, the vibration generated by the movement of the foot massage assembly is mostly absorbed by the four fasteners 26 and is not completely transmitted to the lower cover assembly due to the rigid connection, thereby absorbing the vibration again and reducing noise.

Although the present disclosure has been described in detail with general description and specific embodiments above, on the basis of the present disclosure, some modifications or improvements may be made thereto, which are obvious to those skilled in the art. Therefore, these modifications or improvements made without departing from the spirit of the present disclosure shall fall within the protection scope of the present disclosure.

Terms such as “upper”, “lower”, “left”, “right”, “middle”, etc., referred to in the specification are merely clear for ease of description, and are not intended to limit the scope of implementation of the present disclosure. Changes or adjustments of the relative relationship thereof are considered to fall within the scope of implementations of the present disclosure without substantially changing the technical content.

What is claimed is:

1. A foot massager, comprising:
an upper cover assembly;
a lower cover assembly; and
a foot massage assembly;
wherein the upper cover assembly is fixed to the lower cover assembly through screws, and the foot massage assembly is disposed between the upper cover assembly and the lower cover assembly;
wherein the foot massage assembly comprises a brushless motor, a motor shaft, eccentric wheels, upper connecting rods, lower connecting rods, counterweight blocks and massage heads;
wherein the motor shaft is rotatably connected to the brushless motor, the eccentric wheels are sleeved on the motor shaft, each of the eccentric wheels comprises a first eccentric section and a second eccentric section; an eccentric direction of each first eccentric section is opposite to an eccentric direction of the second eccentric section, each of the upper connecting rods is installed on a corresponding first eccentric section, each of the lower connecting rods is installed on a corresponding second eccentric section, each of the massage heads is installed on an upper end of a corresponding one of the upper connecting rods, and each of the counterweight blocks is installed on a lower end of a corresponding one of the lower connecting rods.

2. The foot massager according to claim 1, wherein the upper cover assembly comprises an upper cover, a top cover, a left foot pad, a right foot pad, a control panel, a display patch, and a light guiding piece, and the foot massager further comprises an ornament and a stainless steel trim;
wherein the upper cover is installed on an upper end of the lower cover assembly, the top cover is buckled with the upper cover, the left foot pad and the right foot pad are installed in the top cover, the control panel and the display patch are sequentially disposed on a top surface of the top cover from bottom to top, the light guiding piece is disposed around the display patch, the ornament and the stainless steel trim are installed on one side of the upper cover assembly, and the stainless steel trim defines round holes configured to transmit sound.

3. The foot massager according to claim 2, wherein the lower cover assembly comprises a lower cover, a shockproof base, anti-friction pads, a main board, a BLUETOOTH board, a speaker, and a direct current (DC) power socket;
wherein the lower cover is buckled with the upper cover, the shockproof base is installed on a lower surface of the lower cover, the shockproof base is of an integrally formed structure and is made from rubber, the anti-friction pads are installed on a bottom portion of the shockproof base, and the main board, the BLUETOOTH board, the speaker, and the DC power socket are installed on the lower cover.

4. The foot massager according to claim 3, wherein the foot massage assembly further comprises a motor seat, a motor fixing piece, vibration mechanisms, bearing seats, connecting rod rubber pieces, and heating assemblies;
wherein the brushless motor is installed on the motor seat through the motor fixing piece, the vibration mechanisms are installed on the motor seat, two ends of the motor shaft of the brushless motor are respectively rotatably connected to the bearing seats, the bearing seats are respectively installed in the vibration mechanisms; upper ends of the upper connecting rods and lower ends of the lower connecting rods are respectively installed in the vibration mechanisms through the connecting rod rubber pieces, the heating assemblies are respectively installed on the upper ends of the upper connecting rods, and the heating assemblies are respectively located at lower portions of the massage heads.

5. The foot massager according to claim 4, wherein each of the heating assemblies comprises an upper heating sheet fixing piece, a heat-conducting copper sheet, a middle heating sheet fixing piece, a heating sheet, and a lower heating sheet fixing piece;
wherein in each of the heating assemblies, the middle heating sheet fixing piece thereof is fixed on a corresponding one of the upper connecting rods, the heating sheet thereof is fixed to the middle heating sheet fixing piece thereof, the heat-conducting copper sheet thereof is disposed on the heating sheet thereof, the upper heating sheet fixing piece thereof is disposed between the heating sheet thereof and a corresponding one of the massage heads, and the lower heating sheet fixing piece thereof is disposed between the heating sheet thereof and a corresponding one of the connecting rod rubber pieces.

6. The foot massager according to claim 5, wherein the foot massage assembly further comprises rubber fixing pieces, the connecting rod rubber pieces respectively disposed on the upper connecting rods are respectively installed on upper portions of the vibration mechanisms through first rubber fixing pieces, and the connecting rod rubber pieces respectively disposed on the lower connecting rods are respectively installed on lower portions of the vibration mechanisms through second rubber fixing pieces.

7. The foot massager according to claim 6, wherein the foot massage assembly further comprises bearing rubber sleeves and motor bearings, wherein the two ends of the motor shaft are respectively installed in the bearing seats through the motor bearings; and each of the bearing rubber sleeves is sleeved on an outer ring of a corresponding one of the motor bearings and are sleeved in a corresponding one of the bearing seats.

8. The foot massager according to claim 7, wherein the foot massage assembly further comprises sealed bearings, first bearing fixing pieces, second bearing fixing pieces, and pressing plates;
wherein the sealed bearings comprise first sealed bearings and second sealed bearings;
wherein each of the upper connecting rods is installed on the corresponding first eccentric section through corresponding first sealed bearings, and a first pressing plate of the pressing plates presses the corresponding first sealed bearings disposed in each of the upper connecting rods on an inner side surface of each of the upper connecting rods;
wherein each of the lower connecting rods is installed on the corresponding second eccentric section through corresponding second sealed bearings, the corresponding second sealed bearings are disposed in each of the lower connecting rods, and a second pressing plate of the pressing plates presses the corresponding second sealed bearings on an inner side surface of each of the lower connecting rods;
wherein each of the first bearing fixing pieces abuts against inner side surfaces of the corresponding first sealed bearings disposed in each of the upper connecting rods; and each of the second bearing fixing pieces abuts against inner side surfaces of corresponding second sealed bearings disposed in each of the lower connecting rods.

9. The foot massager according to claim 8, wherein the foot massage assembly further comprises elastic pins, and the eccentric wheels are limited on the motor shaft through the elastic pins respectively.

10. The foot massager according to claim 9, wherein the foot massage assembly further comprises fasteners, the fasteners are integrally formed rubber structures, the fasteners are respectively installed on end portions of the vibration mechanisms, and the fasteners are hung in the lower cover.
